(19) United States
(12) Patent Application Publication
(10) Pub. No.: US 2026/0034299 A1
(43) Pub. Date: Feb. 5, 2026

(54) INTEGRATED CAPACITIVE FLOWMETER FOR INFUSION PUMP

(71) Applicant: CareFusion 303, Inc., San Diego, CA (US)

(72) Inventor: Vadym SOLOGUB, Limerick (IE)

(21) Appl. No.: 18/792,863

(22) Filed: Aug. 2, 2024

(51) Int. Cl. A61M 5/168; A61M 5/14; A61M 5/142

(52) U.S. Cl. CPC A61M 5/16886; A61M 5/14228; A61M 5/16809; A61M 2005/1401; A61M 2202/0007; A61M 2202/04; A61M 2205/0216; A61M 2205/0233; A61M 2205/10; A61M 2205/3327; A61M 2205/3334; A61M 2205/3379; A61M 2205/50; A61M 2205/60

(57) ABSTRACT

An integrated capacitive flowmeter for an infusion pump is disclosed. A conductive plate is attached to a hammer element that moves according to a cam motion in a pumping mechanism of the pump to periodically compress an flexible infusion line loaded in the pumping mechanism. A processor measures a first capacitance between the conductive plate coupled to the hammer element and a conductor opposite the flexible infusion line when the flexible infusion line is loaded between the conductive plate and the conductor in the pumping mechanism. A volume of the fluid within the flexible infusion line during the filling phase is then determined based on the measured capacitance, and a flow rate determined based on the determined volume and a cycle speed of the pump. The speed of the pump can then be adjusted based on a difference between the determined flow rate and a programmed flow rate.

### BACKGROUND

Modern infusion pumps are capable of delivering fluid in a range of 0.1 mL per hour up to thousands of mL per hour. However, there are no known commercially available flowmeters which cover with reasonable accuracy such a broad range, and accuracy is further diminished in low flow rates (e.g., in a range of single milliliters per hour and below). Moreover, conventional flowmeters typically operate using the principal of thermal transfer and require direct contact with fluid, which makes them expensive and susceptible to contamination. A typical dynamic range of measurement may be at best 10:1, while required ranges often reach 10000:1. Therefore, the conventional range of flowmeter applications remains limited, with almost all infusion pumps operating under flow measurement limitations while in open loop control.

Conventional infusion pumps typically rely on a calibration of the pump's under-hammer chamber volume for a number of cycles performed. Multi-point calibration techniques may be utilized at different infusion rates, as inertial effects change the effective volume per cycle (e.g., typically decreasing it at higher speeds). However, calibration may still remain inconsistent and unstable, as it can be affected by the material make up of the infusion set used, the presence of air in set, changes in fluid viscosity, in-set pressure, variations in mechanical setup, and various other environmental and operation factors. As a result, the typical long-term accuracy of an infusion pump may reach only several percent, quickly degrading down to a dozen or more of percent accuracy when the flow rate is set far enough from its calibration point.

### SUMMARY

There is a need for a flowmeter that can accurately measure flow rate across the broad range of fluid flow provided by modern infusion pumps. Accordingly, the subject technology provides an apparatus and method for indirect high-dynamic range flow measurement, mitigating many operational and environmental influencing factors by determining an actual per-cycle fluid volume delivered to a recipient. This enables a control loop to close while significantly decreasing (in some cases fully eliminate) any disturbing factors which may further affect the accuracy of the measurement.

According to various implementations, a system for measuring a flow rate of an infusion pump is disclosed. The system comprises at least one hammer element of a pumping mechanism of the infusion pump, the hammer element configured to move according to a cam motion of a cam associated with the pumping mechanism, to apply a periodic compression to a flexible infusion line when the flexible infusion line is loaded in the pumping mechanism; and one or more processors operatively coupled to the conductive plate and configured to: cause the at least one hammer element to move according to the cam motion and a cycle speed to periodically compress the flexible infusion line to fill the flexible infusion line with a fluid from a fluid reservoir during a filling phase of the cam motion and to deliver the fluid downstream of the hammer element during a delivery phase of the cam motion; measure, during the cam motion, a first capacitance between the conductive plate and a conductor opposite the flexible infusion line; determine, based on the measured first capacitance, a volume of the fluid within the flexible infusion line during the filling phase; determine a flow rate based on the determined volume and the cycle speed; and adjust a speed of the infusion pump based on a difference between the determined flow rate and a programmed flow rate.

According to various implementations, a method for measuring a flow rate of an infusion pump comprises causing at least one hammer element of a pumping mechanism of an infusion pump to move according to a cam motion to periodically compress an flexible infusion line loaded in a pumping mechanism of the infusion pump, according to a cycle speed, to fill the flexible infusion line with a fluid from a fluid reservoir during a filling phase of the cam motion and to deliver the fluid downstream of the hammer element during a delivery phase of the cam motion; measuring, during the cam motion, a first capacitance between a conductive plate coupled to the hammer element and a conductor opposite the flexible infusion line when the flexible infusion line is loaded in the pumping mechanism, between the conductive plate and the conductor; determining, based on the measured first capacitance, a volume of the fluid within the flexible infusion line during the filling phase; determining a flow rate based on the determined volume and the cycle speed; and adjusting a speed of the infusion pump based on a difference between the determined flow rate and a programmed flow rate.

Accordingly, the subject technology allows indirect flow and volume measurement, does not require contact with fluid, has a contamination-resistant and cost-efficient mechanical design, and requires no special materials and/or components. The method of measurement is also computationally light, which can be ran along with other tasks on low-end microcontrollers and while under closed-loop fluid delivery control. Accordingly both total volume and momentary flow rate inaccuracy is reduced, while operating under a wide range of flow rates, starting form 10ths of microliters and up to several litters per hour. Moreover, the disclosed system and method is self-calibrating and self-adjusting, allowing for the use of infusion sets made of different materials and use of fluids with various viscosity, while eliminating the need for expensive and frequent calibrations by qualified personnel.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts a perspective view of an example infusion device showing an infusion set in place within the infusion device, according to various aspects of the subject technology.

FIG. 2A depicts an example pumping mechanism of an infusion pump including two occluder valves, according to various aspects of the subject technology. FIG. 2B depicts an example delivery pattern over time for the example pumping mechanism of FIG. 2A.

FIGS. 3A-C depict example states of a pumping mechanism of an infusion pump, according to various aspects of the subject technology.

FIG. 4 depicts a pumping mechanism configured with an integrated capacitive flowmeter according to various aspects of the subject technology.

FIGS. 5A-C depict example parameters of the disclosed integrated capacitive flowmeter, according to various aspects of the subject technology.

FIG. 6 depicts example measurements of capacitance over multiple pumping cycles, according to various aspects of the subject technology.

FIG. 7 depicts a linear relationship between capacitance and filling factor dependency when capacitance measurement are integrated over each pumping cycle.

FIG. 8 depicts an example process for measuring a flow rate of an infusion pump with a capacitive flowmeter integrated in an infusion pump, according to aspects of the subject technology.

FIG. 9 depicts an example pumping mechanism including an attached conductive plate and operatively coupled processor, according to various aspects of the subject technology.

FIG. 10 is a conceptual digital circuit diagram of an example processor and related circuitry for a capacitive flowmeter, according to various aspects of the subject technology.

FIG. 11 is a conceptual diagram illustrating an example electronic system for measuring a flow rate of an infusion pump with a capacitive flowmeter integrated in an infusion pump, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology includes a system and method for indirect high-dynamic range fluid flow measurement using an integrated capacitive flowmeter. An infusion pump includes at least one hammer element configured to move according to a cam motion to apply a periodic compression to a flexible infusion line loaded in a pumping mechanism of the pump. According to various implementations described herein, a conductive plate is coupled to the hammer element, between the hammer element and the flexible infusion line when the flexible infusion line is loaded in the pumping mechanism. The hammer element is caused to move according to the cam motion and a cycle speed to periodically compress the flexible infusion line to fill the flexible infusion line with a fluid from a fluid reservoir during a filling phase of the cam motion and to deliver the fluid downstream of the hammer element during a delivery phase of the cam motion. During this motion, a processor measures a capacitance between the conductive plate and a conductor opposite the flexible infusion line, and determines, based on the measured first capacitance, a volume of the fluid within the flexible infusion line during the filling phase. A flow rate may then be determined based on the volume and a cycle speed, and a speed of the infusion pump may be adjusted based on a difference between the determined flow rate and a programmed flow rate.

The configuration of the subject technology is self-tuning and self-calibrating and greatly simplifies and reduces the need for service and maintenance of the pump into which it integrates. The flowmeter improves flow stability, is cost-efficient, low power, durable, easily adopted to any existing linear pump mechanism, and works in a wide range of flows from 0 to thousands of ml/Hour.

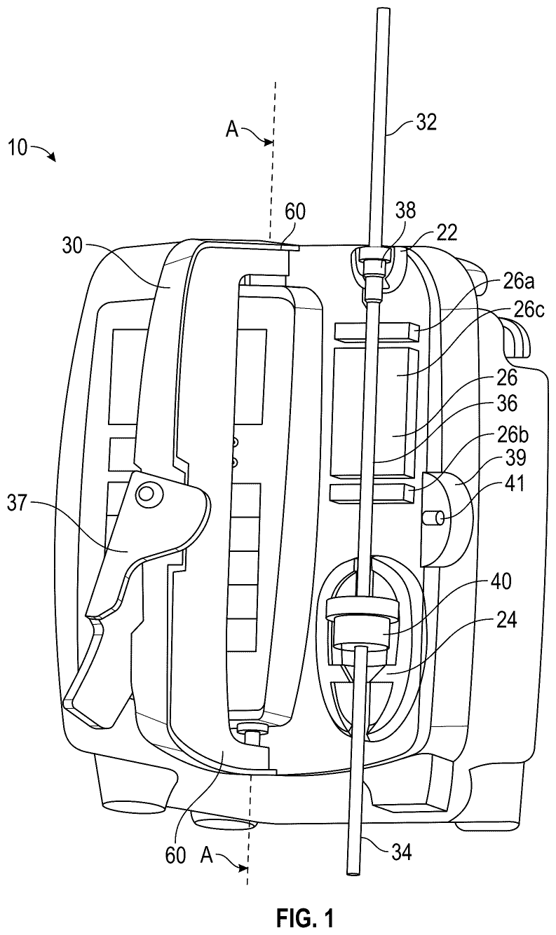

FIG. 1 depicts a perspective view of an example infusion device showing an infusion set in place within the infusion device, according to various aspects of the subject technology. An infusion system for parenteral infusion of a medical fluid to a patient comprises a pump unit, a major part of which comprises a housing which accommodates, in manner known per se, a cam system (not shown) controlling one or more hammers (or, e.g., fingers) of a peristaltic pumping mechanism, an electric motor and associated gearing, driving said cam mechanism, and further accommodates electronic control and processing circuitry for controlling such motor and processing signals from pressure sensors etc. provided on the unit. The pump unit, as shown, may also comprise an electronically operated display, an alarm light, an input keyboard or other manually operated controls, all in manner known per se.

As shown in FIG. 1, the infusion device 10 may include a door 30 or face plate which may be opened to reveal the internal loading mechanism for an infusion set. Within the housing of the infusion device (e.g., behind the door or face place), the infusion device includes a pumping segment 36 including a group of serially-aligned pumping elements configured to compress an elongated compressible channel of the infusion set, when loaded within the pumping segment. The pumping segment includes a group of serially-aligned pumping elements (e.g., occluders and/or hammer(s)) configured to compress the elongated compressible channel (e.g., an IV tubing segment) loaded within the pumping segment.

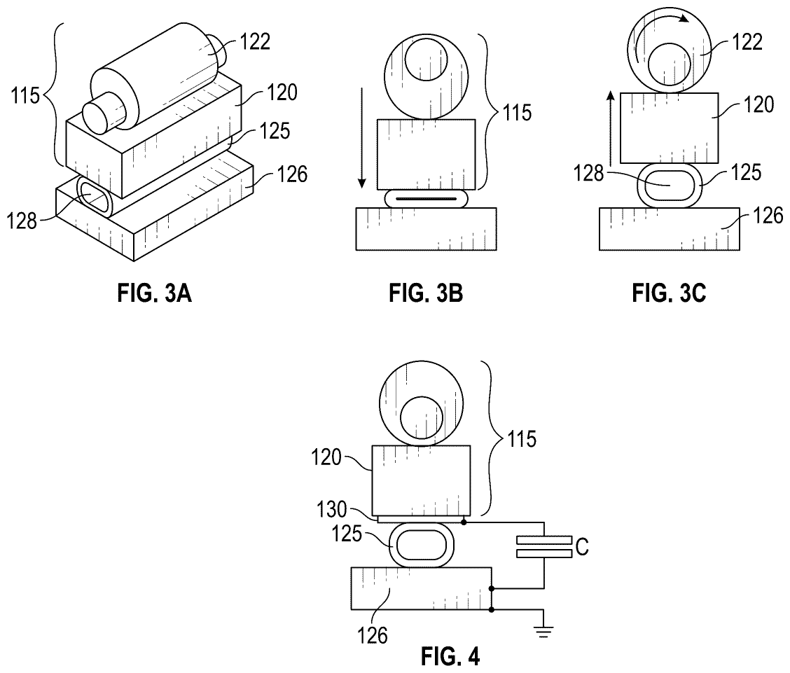

The infusion set includes upper and lower sections 32 and 34 respectively of a tubing, a pumping segment 36 including an intermediate section of resiliently compressible tubing, for example of silicone rubber and, in some implementations, upper and/or lower fittings 38 and/or 40 via which the intermediate tubing section 36 may be connected respectively with a respective upper line 32 and with the lower line 34. In use, each upper line 32 extends upwardly to a source of the medical fluid to be administered whilst the lower line 34 extends from the infusion pump to an infusion needle or the like inserted into the patient. In use, the pumping segment 36 of infusion set is extended across the face or deck of the pump unit so that the fittings 38 and 40 are received in respective brackets 22 and 24 respectively and so that each tubing segment 202, 204 extends over a respective peristaltic assembly 26 as illustrated in FIG. 3. In the depicted example, the infusion set is fitted in place in this fashion whilst the door 30 is in the open position. After the infusion line has been so fitted, the door 30 may be moved to the closed position and is secured by a catch 37 which may include a lever mounted on the outer edge of the door.

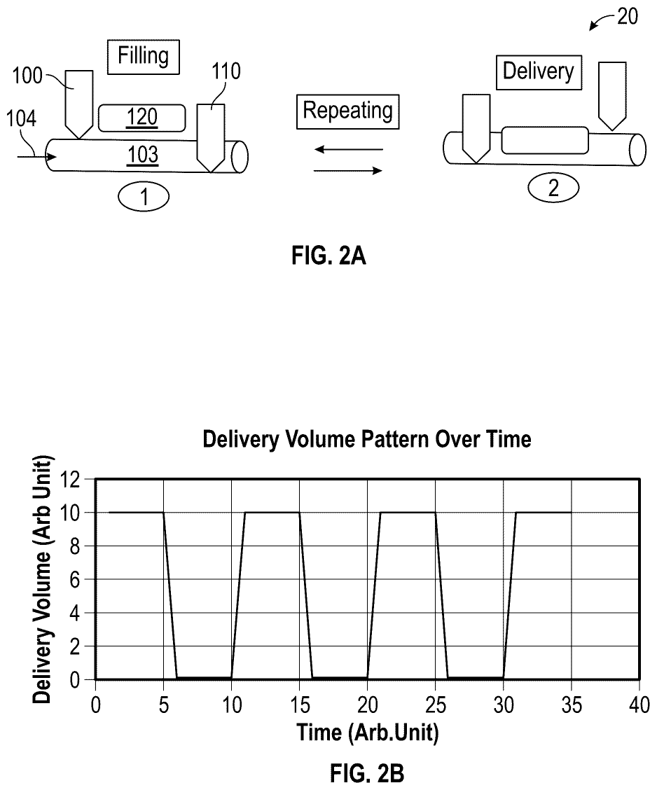

The peristaltic assembly 26 includes one or more hammers/fingers that are moveable by a cam system (not shown) inwards and outwards from the face or deck 20 of the pump to compress a respective tubing segment against a counter surface or anvil to propel fluid within the infusion line. In order to make it easier to maintain sterile conditions, the hammers/fingers may be covered by a thin flexible membrane, sealed at its edges with respect to the deck. The hammer(s) of the peristaltic assembly 26 periodically presses the flexible resilient tubing against a counter surface or plate which may be positioned on a side of the tubing section 36 opposite of the peristaltic assembly 26, for example, on an inner portion of the door 30. In the example pump shown, each peristaltic assembly includes an upper occluder 26 a and a lower occluder 26 b which are of a relatively limited extent in the longitudinal direction of the infusion line, and an intermediate hammer 26 c, between the upper and lower occluders and which hammer 26 c is extended or elongated in the longitudinal direction of the infusion line. In operation, assuming the fluid is to be propelled downwards, as viewed in FIGS. 2 and, along the infusion line, the peristaltic assembly performs a repeating cycle in which, with the intermediate pad 26 c spaced from the counter surface, the upper occluder 26 a presses the flexible tube against the counter surface or anvil to close the tube at the location of the upper occluder 26 a, the lower occluder 26 b is then withdrawn from the counter surface to open the tube at the location of the lower occluder 26 b, then the hammer 26 c is moved towards the counter surface to drive the fluid in the tube adjacent the intermediate pad 26 c downward along the tube, then the tube is pinched closed again between the lower occluder 26 b and the counter surface, then the upper occluder 26 a is withdrawn from the counter surface and the hammer 26 c withdrawn from the counter surface to draw fresh fluid into the part of the tube adjacent the hammer 26 c.

FIG. 2A depicts an example pumping mechanism 20 of an infusion device 10 including two occluder valves 100, 110 (or 26a, 26b), according to various aspects of the subject technology. A typical peristaltic medical pump for IV infusion delivery has two occluders, a first occluder 100 located upstream and a second occluder 110 located downstream, with a hammer 120 (or 26c) in between. The occluders and hammer coordinate with each other in programmable, sequential steps, controlled by a cam shaft to have two phases: 1) a filling phase, and 2) a delivery phase. The occluders move fluid in a tubing 103 by sequentially compressing the tubing, thereby causing a flow in a direction 104 according to the particular compression sequence of the occluders.

During the medication infusion process, in the filling phase, the upstream occluder 100 lifts to suck the medication into the tubing segment, which creates a pause, followed by the delivery phase to push the fluid out. These sequences can repeat through multiple cycles. To specify, when the plunger of a single plunger/tubing design is lifted from the tubing segment during the filling phase, there will be a disruption in the continuous infusion process. As a result of using this design, fluid delivery may be performed in a pulse patten as shown in FIG. 2B.

FIGS. 3A-C depict example states of a pumping mechanism of an infusion pump, according to various aspects of the subject technology. The depicted pumping mechanism 115 includes a camshaft 122, driven by a motor (not shown), a hammer 120 (e.g., of peristaltic assembly 26), actuated by a camshaft 124, an administration set 125 (including, e.g., sections 32, 34, and/or 36), and the plate 126 (usually part of the infusion pump door 30), against which the hammer 120 acts. As shown in FIGS. 1 and 2, valves 26 a/b may surround that mechanism, ensuring unidirectional fluid flow.

In accordance with FIGS. 2A and 2B, a pumping process includes multiple cycles, where each cycle is formed by one full revolution of camshaft 122. For exemplary purposes, as depicted in FIG. 3B, a cycle may start with the hammer 120 in its bottom dead center, fully squeezing the pumping segment 36 of infusion set 126, such that the latter does not have free internal volume to contain either fluid or air. Next, as shown in FIG. 3C, the motor rotates and the hammer moves away from the set, releasing pressure on the set, and the pumping segment of the infusion set expands due to its material properties (e.g., a silicon rubber). As the set expands, it creates a vacuum and sucks in fluid from the fluid reservoir upstream of the pump. Once the hammer 120 reaches its top dead center, as depicted in FIG. 3C, the maximum amount of fluid is already in the set. As the camshaft keeps rotating, the cam starts putting pressure on the hammer again. The hammer presses against the set, as depicted in FIG. 3B, and squeezes out the contained fluid in the direction downstream of the pump (e.g., into a patient). Once the hammer reaches its bottom dead center, and the fluid expelled downstream, a new cycle starts again.

The delivered fluid volume per cycle (e.g., under ideal conditions) is equal to the internal volume 128 of the pumping segment 36 at its fully expanded state, which is observed when the hammer is at its top dead point, as indicated in FIG. 3C

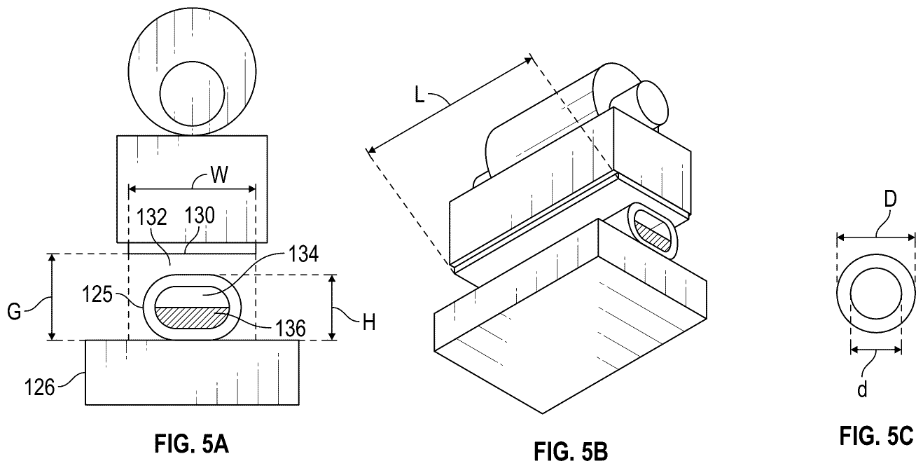

FIG. 4 depicts a pumping mechanism configured with an integrated capacitive flowmeter according to various aspects of the subject technology. A conductor element such as a thin metal plate 130 is attached underneath the hammer 120 of an infusion pump. According to various implementations, the hammer 120 is made of insulation material, typically any durable plastic, which is the case for most of modern pumps. However, the plate 130 may be any thickness and may be formed of any conductive material. For example, the plate may be a glued-on metal foil, or metallized surface of the hammer itself. With reference to FIG. 1, the plate 130 may be implemented as the previously described thin flexible membrane of peristaltic assembly 26 (covering the hammer 120). As will be described further, a flowmeter is constructed by way of measuring the capacitance of a flat two-plate capacitor C formed by the hammer's plate 130 and an opposing conductor 126 (e.g., a grounded metal door of the infusion pump), with the administration set 125 positioned therebetween.

According to the implementation of FIG. 4, the capacitance C is equal to:

$$C = {ε}_{0} \times ε \times \frac{W \times L}{G} , \qquad ( 1 )$$

where W is plate width, L is plate's length (depth), G is gap between the plate and the door, ε₀ is constant 8.854E−12 F/m, and ε is dielectric constant of material between the plate 130 and the conductor 126 (e.g., the platen or plate within the door of the pump).

Considering that W, L, and G are constants, defined by system's design, and depending on fluid presence or absence, equation (1) can be rewritten as:

$$C = ε \times A , \qquad ( 2 )$$

where A is a constant system-dependent factor.

According to various implementations, there are two occluder elements (See e.g., FIG. 1); one for the upper occluder 26 a, and one for the lower occluder 26 b. As described previously, the hammer element 120 is configured to move according to a cam motion of a cam 122 to apply a periodic compression to a flexible infusion line 125 when the flexible infusion line 125 is placed between the Hammer element 120 and the plate assembly 126 (also termed “platen”). In this regard, the cam motion oscillates the hammer element 120 to move a fluid within the flexible infusion line by periodically compressing the flexible infusion line.

In some implementations, compression springs (not shown) may apply a constant force to the hammer element 120, forcing it against the plate assembly 126, while the cam 122 applies a force at predetermined intervals in an opposite direction, moving the portion of the hammer element 120 responsible for compressing the infusion line 125 away from the plate assembly. In other implementations, the compression springs may force the hammer element 120 away from the plate assembly, while the cam moves the hammer element toward the plate assembly 126 to compress the infusion line 125. Each cam 122 may be elliptical in shape and may rotate on an axis off center of the ellipse.

The conductive element or plate 130 is coupled to the hammer element 120, between the hammer element and the flexible infusion line 125, as shown. The capacitance C is measured between the conductive plate 130 and the conductive platen 126 during operation. As will be described further, the delivered fluid volume per cycle (within the internal volume 128) is determined, based on the measured capacitance, and a flow rate of the fluid determined based on the determined volume and cycle speed of the pumping segment.

While the subject technology described herein is described with regard to hammer elements, the subject technology is equally applicable to other systems which provide a compression to a fluid filled tubing. For example, a peristaltic pumping mechanism having a multitude of fingers may be operated using the subject technology in the same way previously described.

FIGS. 5A-C depict example parameters of the disclosed integrated capacitive flowmeter, according to various aspects of the subject technology. According to various implementations, the capacitance between the conductive plate 130 and the conductive platen 126 is based on a dielectric function of one or more factors associated with a gap between the conductive plate 130 and the conductor 126 opposite the flexible infusion line 125. Factors may include the dimensions of the gap itself, a dielectric constant associated with the gap, for example, based on air within the gap, a dielectric constant associated with the fluid that is filled within the flexible infusion line during the filling phase, and/or a dielectric constant associated with a material of the flexible infusion line.

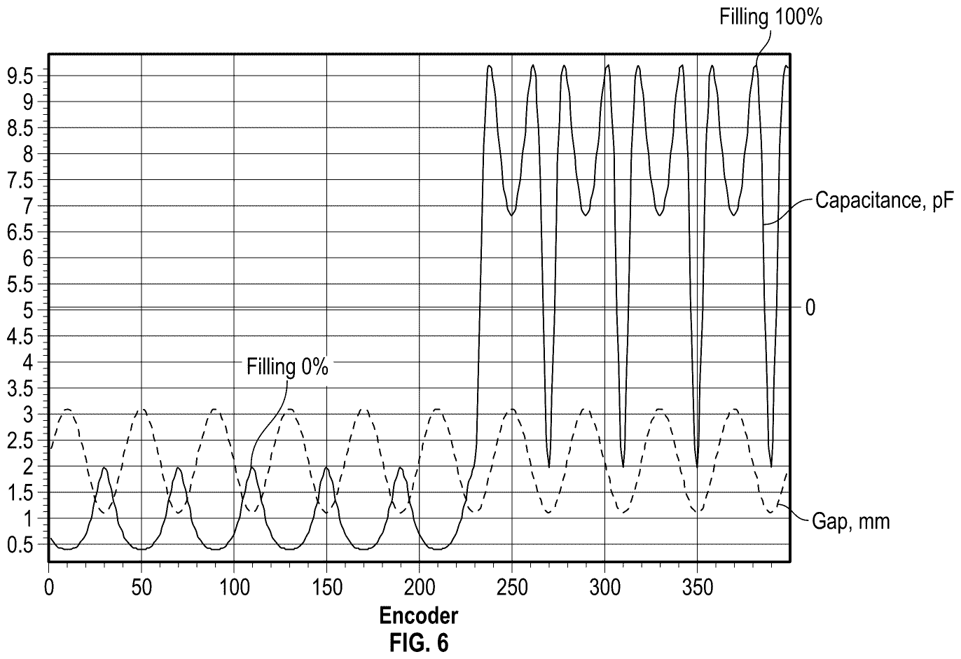

Types of dielectric inside the plate-door gap may include one or more of: air 132 around the infusion set 125, eventual air 134 inside the set, the administration set 125 itself, and fluid 136 inside the set. The set's height H may not necessarily be equal to gap G. According to some implementations, the equivalent dielectric constant & of all the dielectrics in the gap can be estimated as followed:

$${ε}_{e q} = \frac{{ε}_{a i r} \times {s}_{a i r} + {ε}_{s e t} \times {S}_{w a l l s} + {S}_{i n t} \times ( 1 + ( {ε}_{f l uid} - 1 ) \times {K}_{f i l l} )}{{S}_{g a p}} , \qquad ( 3 )$$

where:
εₐᵢᵣ=dielectric constant of air, equal to 1
εₛₑₜ=dielectric constant of set's material, typically 3 for silicone
ε_{fluid}=dielectric constant of fluid, typically 80 for water.
Sₐᵢᵣ=area of gap's cross-section, occupied by air around the set
S_{walls}=area of cross-section of set's wall
Sᵢₙₜ=internal area of set's cross-section.
S_{gap}=total area of gap's cross-section (enclosed in dashed lines in FIG. 6), W×G
K_{fill}=set's fluid filling factor, between 0 and 1. Where 0 corresponds to no fluid, and 1 corresponds to no air.

Considering S_{walls}=const, Sₐᵢᵣ«S_{gap}, εₐᵢᵣ«εₛₑₜ«ε_{fluid}, and ε_{fluid}»1, we can neglect first three terms in (1), rewriting it as followed:

$${ε}_{e q} \approx \frac{{S}_{i n t}}{{S}_{g a p}} \times {ε}_{fluid} \times {K}_{f i l l} + B \qquad ( 4 )$$

Where B is a constant system-dependent parameter.

By combining (2) and (4) and joining constant terms:

$$C = \frac{{S}_{i n t}}{{S}_{g a p}} \times {ε}_{f l u i d} \times {K}_{f i l l} \times A + N , \qquad ( 5 )$$

Or, finally

$$C = M \times {K}_{f i l l} + N . \qquad ( 6 )$$

From (6), the variables can be broken down to constants M and N, and the measured capacitance C is thus shown to be linearly proportional to filling factor K_{fill}, that is,

$$Δ C = P \times Δ {K}_{f i l l} , \qquad ( 7 )$$

where P is a constant factor of proportionality.

Considering that fluid volume, delivered per pumping cycle (single shaft rotation) is equal to:

$${V}_{c y c l e} = {S}_{int} \times L \times {K}_{fill} = {Q}_{c onst} \times {K}_{f i l l} , \qquad ( 8 )$$

where Q_{const} is a system-dependent constant conversion factor, the following fluid flow measurement method is proposed:

1. At the beginning of priming, before infusion starts, the system measures capacitance Cₘᵢₙ of empty set, corresponding to filling factor K_{fill}=0.

2. During priming, the system determines a maximum measured capacitance Cₘₐₓ, and assumes it corresponds to fully filled with fluid set, i.e. to K_{fill}=1.

3. During infusion, knowing that C and K_{fill} are linearly proportional (see equation (7)), actual K_{fill} values are calculated by linear interpolation:

$${K}_{f i l l} = \frac{{C}_{m e a s u r e d} - {C}_{min}}{{C}_{max} - {C}_{min}} . \qquad ( 9 )$$

4. Then it determines per-cycle volume V by using equation (8) and then final fluid flow, as:

$$Flow = V \times R , \qquad ( 10 )$$

where R is camshaft's angular speed, revolutions per time unit.

5. During infusion, the system may continuously self-calibrate with aid of other sensors (for instance, air-in-line) by adjusting Cₘᵢₙ and Cₘₐₓ, compensating this way for changes in environmental and operational conditions, and further improving measurement accuracy.

FIG. 6 depicts example measurements of capacitance over multiple pumping cycles, according to various aspects of the subject technology. In testing the disclosed system, it has been found that stable results can be achieved by integrating measured capacitance over each pumping cycle (e.g., a camshaft revolution). Such integration may further desensitize the system to measurement noise, mechanical tolerances, and operating conditions,—preserving high linearity at the same time.

In the depicted example, capacitance variation is plotted for flexible infusion line 125 with internal diameter d (FIG. 5C) of 3 mm, set's external diameter D of 4.1 mm, filling factors of 1 (set entirely filled with fluid) and 0 (set is entirely filled with air), total hammer's stroke of 2 mm, and hammer's plate of 6×14 mm.

During operation or during a calibration of the system, the collection of measurements and execution of one or more steps may be controlled by a microcontroller or other processing device specifically configured by machine-executable instructions stored in a data storage device. For example, if the initial capacitance values indicate an unexpected pattern indicative of or otherwise satisfying a threshold for a low filling capacity (K_{fill}), the microcontroller may transmit a control message to the pump to jog the pump motor through a sequence of positions in an attempt to correct the filling of the infusion set. The microcontroller may store a log of the capacitance and determination results in association with an identifier for the pump to provide an auditable record of the pump state and/or tests performed.

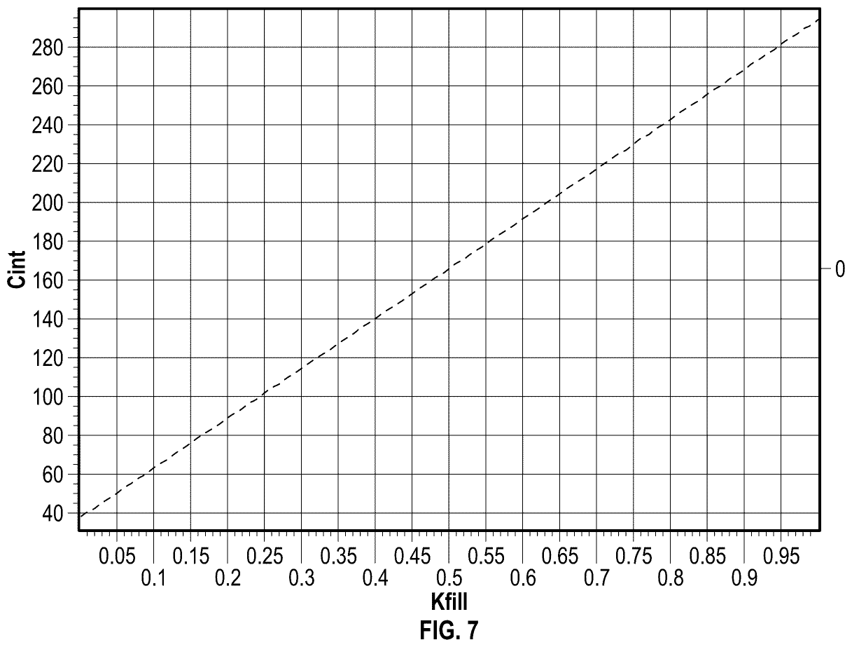

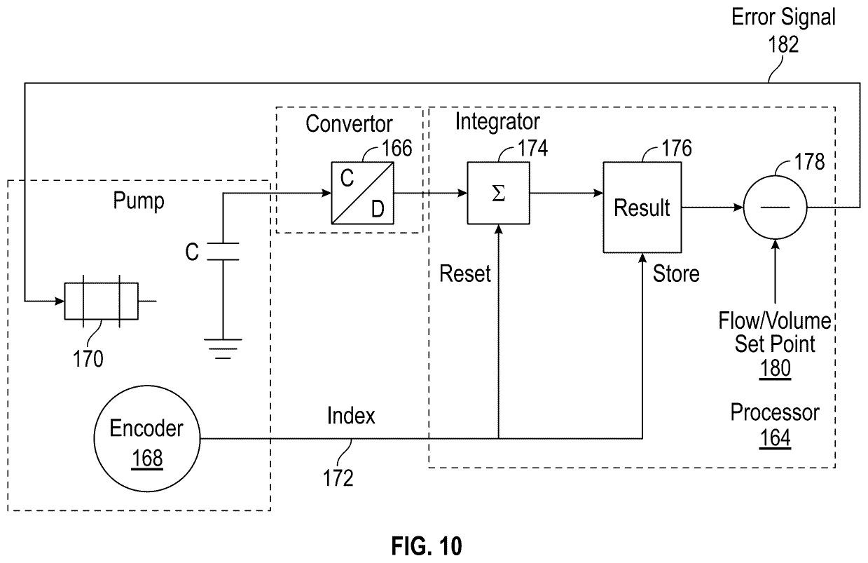

FIG. 7 depicts a linear relationship between capacitance and filling factor dependency when capacitance measurement are integrated over each pumping cycle. Here Cᵢₙₜ is integrated over single pumping cycle capacitance, pF, received in preferred embodiment as per FIG. 10. In this particular case, equation (9) will use Cₘᵢₙ=35 and Cₘₐₓ=295. The integration of C may provide improved resolution for the determination of K_{fill}. The linear relationship between capacitance and filling factor may be used to provide lookup functionality of K_{fill} based on Cᵢₙₜ.

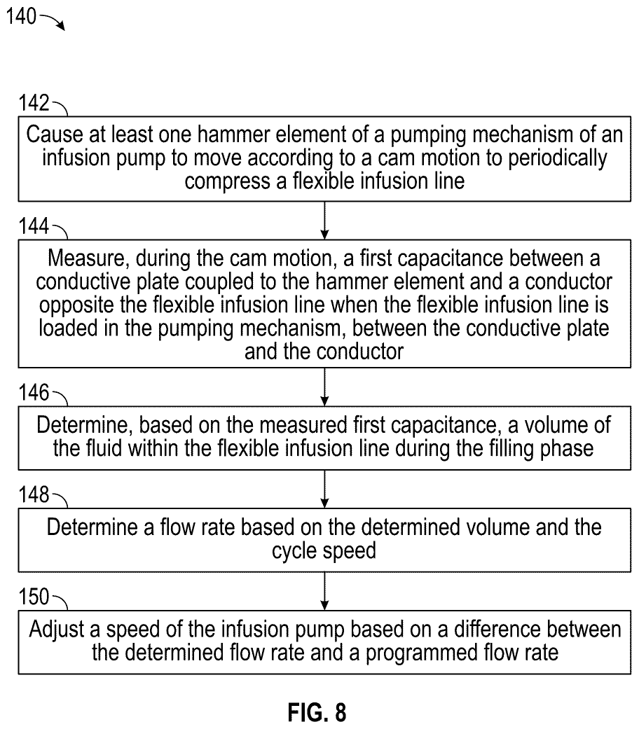

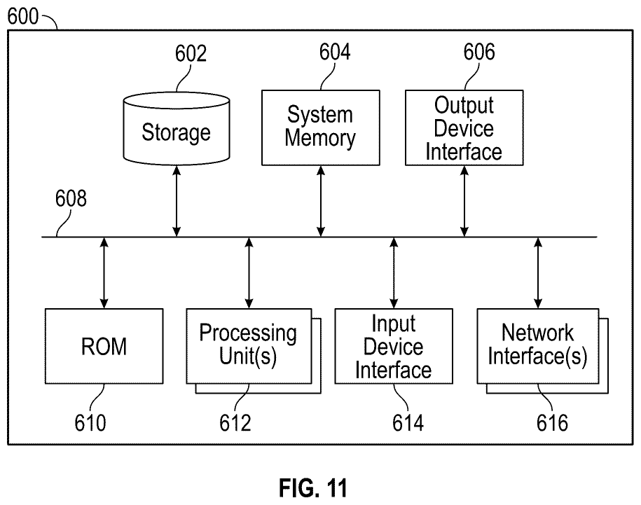

FIG. 8 depicts an example process 140 for measuring a flow rate of an infusion pump with a capacitive flowmeter integrated in an infusion pump, according to aspects of the subject technology. For explanatory purposes, the various blocks of example process 140 are described herein with reference to FIGS. 1 through 7, and the components and/or processes described herein. The one or more of the blocks of process 140 may be implemented, for example, by one or more processors and/or computing devices including, for example, within infusion device 10 (e.g., electronic system 600 and/or processor(s) 612 of FIG. 11). In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 140 are described as occurring in serial, or linearly. However, multiple blocks of example process 140 may occur in parallel. In addition, the blocks of example process 140 need not be performed in the order shown and/or one or more of the blocks of example process 140 need not be performed.

An infusion system (e.g., the infusion pump 10 of FIG. 1) includes at least one hammer element 120 (e.g., 26 of FIG. 1) configured to move according to a cam motion of a cam 122 associated with a pumping mechanism of the infusion pump 10. Accordingly, the pumping mechanism is configured to move the hammer element(s) 120 to apply a periodic compression to a flexible infusion line 125 when the flexible infusion line is loaded in the pumping mechanism. With reference to FIG. 1, loading the flexible infusion line may involve positioning it between the hammer element and a plate assembly in a door 39 of the infusion pump 10.

In the depicted example, a processor(s) causes the hammer element(s) 120 to move according to the cam motion and a cycle speed to periodically compress the flexible infusion line 125 (142). Such action causes the flexible infusion line 125 to fill with a fluid from a fluid reservoir during a filling phase of the cam motion and to deliver the fluid downstream of the hammer element 120 during a delivery phase of the cam motion (e.g., when the lower occluder 26 b of FIG. 1 is in an open state).

According to various implementations, a conductive plate 130 is coupled to the hammer element(s) 120, between the hammer element and the flexible infusion line when the flexible infusion line is loaded in the pumping mechanism. The processor(s) measures, during the cam motion, a first capacitance between the conductive plate 130 and a conductor 126 opposite the flexible infusion line (144). According to various implementations, the plate assembly within the door 30 of the infusion pump 10 may function as the conductor opposite the flexible infusion line. In some implementations, the conductor 126 opposite the flexible infusion line is grounded.

In some implementations, the measured capacitance may be based on a dielectric function comprising a dielectric constant associated with a gap between the conductive plate and the conductor opposite the flexible infusion line, a dielectric constant associated with the fluid that is filled within the flexible infusion line during the filling phase, and/or a dielectric constant associated with a material of the flexible infusion line. As described previously, the dielectric constant associated with the gap may be based on one or more variable parameters (e.g., equation 3), or may be reduced down to one or more constants (e.g., equations 6 or 7).

In some implementations, any one of the constants may be received via input from a user (e.g., at the pump's control panel or via a connected computing device), or from an external system (e.g., from an automated programming message provided by a server). In this regard, the infusion pump 10 may receive an input of an infusion set identifier, and obtain the dielectric constant associated with the material based on the infusion set identifier. Similarly, the infusion pump 10 may receive a fluid identifier; and obtain the dielectric constant associated with the fluid based on the fluid identifier.

In some implementations, the measurements of the capacitance may be sampled over one or more revolutions of the cam 122 and then integrated as Cᵢₙₜ. For example, the capacitance may be sampled over a single revolution and the sampled capacitance integrated over the revolution; e.g., from low dead center to low dead center. Accordingly, the integration and determination of Cᵢₙₜ may be performed when the hammer element is positioned at a low dead center (e.g., nearest the plate assembly 126) during the delivery phase and the flexible infusion line compressed. In some implementations, the capacitance may be measured when the cam is in a position in which the hammer element 120 responsible for compressing the infusion line 125 is furthest away from the plate assembly 126—i.e., high dead center during the filling phase and the flexible infusion line in the most expanded state-when the flexible infusion line is expected to be filled.

The processor(s) then determine, based on the measured capacitance, a volume of the fluid within the flexible infusion line during the filling phase (146).

In some implementations, the volume is determined based on the equation V_{cycle}=Q_{const}×K_{fill}, wherein Q_{const} is a system-dependent constant conversion factor that accounts for properties of the flexible infusion line and a length of the flexible infusion line between the conductive plate and the conductor opposite the flexible infusion line.

In some implementations, the processor(s) first determines a fill factor K_{fill} based on the capacitance C, which is then used to determine the volume. For example, the processor(s) may obtain, from a lookup database (or algorithm), the fill factor based on the measured capacitance; and determine the volume based on a function of the fill factor and one or more predetermined variables. With reference to FIG. 7, the integral of the capacitance may be used to determine or otherwise obtain the filling factor K_{fill}.

In some implementations, the filling factor K_{fill} is determined according to a calibration process. For example, K_{fill} may be determined according to the equation

$${K}_{f i l l} = \frac{{C}_{m e a s u r e d} - {C}_{min}}{{C}_{max} - {C}_{min}} ,$$

wherein Cₘᵢₙ is the initial capacitance when the flexible infusion line is empty and Cₘₐₓ is the primed capacitance when the flexible infusion line is primed with the fluid.

In one example, the calibration process may take place during a priming operation. The clinician may indicate via a control input at an interface of the infusion pump 10 that priming of the flexible infusion line 125 will be initiated. In some implementations, the pump may prompt the clinician to prime the infusion line as part of a programmed infusion. The infusion pump may then automatically measure the initial capacitance when the infusion line is empty. The clinician may then initiate priming and indicate the infusion line has been primed via the control input at an interface of the infusion pump 10. The processor(s) may then measure a primed capacitance when the flexible infusion line is primed with the fluid, and automatically determine a filling factor based on a function of the measured first capacitance, the initial capacitance, and the primed capacitance, according to equation 9. The volume V may then be determined based on a function of the filling factor and one or more predetermined factors, such as is shown in equation 8.

The processor(s) proceeds to determine a flow rate based on the determined volume and the cycle speed (148), and adjust a speed of the infusion pump based on a difference between the determined flow rate and a programmed flow rate (150). In some implementations, the volume is determined for a plurality of cam cycles and then the flow rate determined based on the determined volume for each of the plurality of cam cycles over a given period.

In some implementations, the volume for a given cycle, the filling factor, and the flow rate are determined when the hammer element is positioned at a low/bottom dead center during the delivery phase and the flexible infusion line compressed.

Many of the above-described devices, systems and methods, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

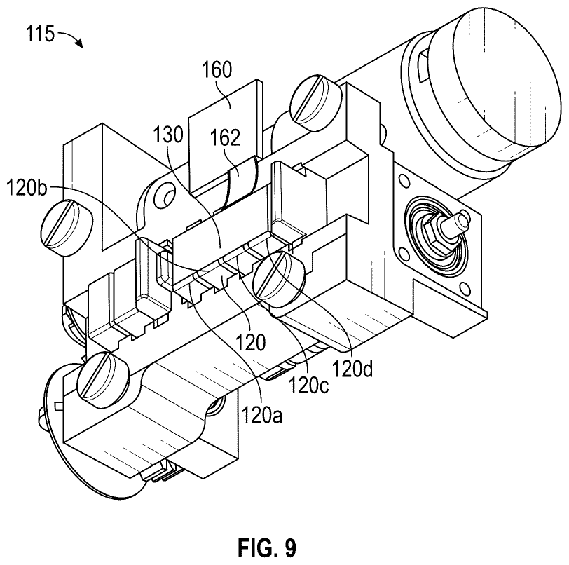

FIG. 9 depicts an example pumping mechanism 115 including an attached conductive plate 130 and an operatively coupled processing circuit 160, according to various aspects of the subject technology. The processing circuit 160 may be responsible for facilitating or performing any of the operations described previously with regard to FIGS. 1-8.

As described previously, the conductive plate 130 is coupled/attached to a hammer element 120. In the depicted example, the conductive plate 130 is a single rigid plate (e.g., a 14 mm×6 mm×0.1 mm stainless steel plate). However, the plate may include a metal foil or other flexible membrane or a metallized surface of the hammer itself.

While the plate 130 may be shown to accommodate one hammer, it is understood that the pumping mechanism 115 may have multiple hammers and/or one or multiple plates. For example, there may be multiple hammer elements (operating as pumping fingers) with a single flexible conductive plate over the top of the hammers such as to allow each hammer element to individually compress a flexible infusion line positioned below the hammer(s). In the depicted example, four elements 120 a-d may be seen. In some implementations, each hammer may have its own conductive plate so that a capacitance of each hammer may be measured between the individual hammer and the conductor opposite the flexible infusion line (e.g., in the pump door).

The conductive plate 130 is connected with flat flexible polyamide cable 162 to the capacitance-to-digital (CDC) convertor's PCB which houses the processing circuit 160 and is fixed to the motor block. The platen in the pumps door (or metal door), which acts as the counterpart conductor for the depicted conductive plate 130 is not shown but can be understood from FIGS. 1 and 4.

FIG. 10 is a conceptual digital circuit diagram of an example processing circuit 160 for a capacitive flowmeter, according to various aspects of the subject technology. The processing circuit 160 may include, for example, a microcontroller 164 that includes an analog-digital (A/D) converter 166 for receiving capacitance values from the previously described capacitor arrangement of FIG. 4. In some implementations, however, the A/D converter 166 may be a separate circuit that is provided alongside of the microcontroller 164 and which feeds its digital input into the microcontroller. The pump's encoder 168 provides further input pertaining to index values of the pump's motor 170 to the microcontroller 164, including the current position of the motor and/or the pump's cam 122 (e.g., for determining the current angle of the cam).

In the depicted example, the measured capacitance C is input into the A/D converter 166, which converts the analogue reading to a digital value, which is then input into the microcontroller 164. Contemporaneous with receiving and converting the capacitance C, the microcontroller 164 may receive an index value 172 from the pump's encoder 168, e.g., as the cam is rotated by the pump's motor 170, thereby enabling the microcontroller 164 to select a value corresponding to a position of the encoder and/or cam.

As described previously, a plurality of capacitance values may be sampled over one or more pumping cycles. For example, a processing algorithm may sample the capacitor values at fixed or variable rate and integrates (2) the sampled value over one pumping cycle (one camshaft revolution). The digital representations of capacitance may then be integrated, by an integrator stage 174 of the microcontroller 164, over a pump cycle determined by way of the encoder index values 172 received from the pump's encoder 168, as described previously.

The filling factor K_{fill} and flow rate are then determined, as described previously, e.g., based on equations (8), (9), and (10), respectively, at a result stage 176 of the microcontroller 164. In some implementations, the filling factor and the flow rate (and volume) are determined when the hammer element is positioned at a low/bottom dead center. The bottom dead center can be determined by the microcontroller 164 based on the value of the encoder index signal.

A comparator stage 178 of the microcontroller 164 may be configured to receive a flow and/or volume set point 180 provided by the infusion pump (e.g., based on input from a user or external system, as previously described). An error signal 182 may then be determined based on a comparison between the flow or volume result from the result stage 176 and the respective flow or volume set point 180. In this regard, the determined flow and volume get compared with required flow/volume set point and the comparison error 182 controls the pumping motor 170, thereby closing the control loop.

When the result is recorded, the integral may be reset, therefore getting ready for next cycle integration. The recorded value may then be used for overall flow calculations/corrections. In some implementations, the integral may be stored and then immediately reset at each bottom dead centre of hammer's position (minimal gap), as described previously.

FIG. 11 is a conceptual diagram illustrating an example electronic system 600 for measuring a flow rate of an infusion pump with a capacitive flowmeter integrated in an infusion pump, according to aspects of the subject technology. Electronic system 600 may be a computing device for execution of software associated with one or more components and processes provided by FIGS. 1 to 10, including but not limited to infusion device 10. Electronic system 600 may be representative of a device used in connection or combination with the disclosure regarding FIGS. 1 to 10. In this regard, electronic system 600 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, a processing unit(s) 612 retrieves instructions to execute and data to process, in order to execute the processes of the subject disclosure. The processing unit(s) 612 can be a single processor or a multi-core processor or multiple processors working together, any of which may include or be implemented by the processing circuit 160 or associated microcontroller 164.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 11, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. A system for measuring a flow rate of an infusion pump, comprising: at least one hammer element of a pumping mechanism of the infusion pump, the hammer element configured to move according to a cam motion of a cam associated with the pumping mechanism, to apply a periodic compression to a flexible infusion line when the flexible infusion line is loaded in the pumping mechanism; a conductive plate coupled to the hammer element, between the hammer element and the flexible infusion line when the flexible infusion line is loaded in the pumping mechanism; and one or more processors operatively coupled to the conductive plate and configured to: cause the at least one hammer element to move according to the cam motion and a cycle speed to periodically compress the flexible infusion line to fill the flexible infusion line with a fluid from a fluid reservoir during a filling phase of the cam motion and to deliver the fluid downstream of the hammer element during a delivery phase of the cam motion; measure, during the cam motion, a first capacitance between the conductive plate and a conductor opposite the flexible infusion line; determine, based on the measured first capacitance, a volume of the fluid within the flexible infusion line during the filling phase; determine a flow rate based on the determined volume and the cycle speed; and adjust a speed of the infusion pump based on a difference between the determined flow rate and a programmed flow rate.

Clause 2. The system of Clause 1, wherein the one or more processors are further configured to: obtain, from a lookup database, a fill factor based on the measured capacitance; and determine the volume based on a function of the fill factor and one or more predetermined variables.

Clause 3. The system of Clause 1, the one or more processors further configured to: measure an initial capacitance when the flexible infusion line is empty; measure a primed capacitance when the flexible infusion line is primed with the fluid; determine a filling factor based on a function of the measured first capacitance, the initial capacitance, and the primed capacitance; and determine the volume based on a function of the filling factor and one or more predetermined factors, wherein the cycle speed comprises a number of cam revolutions per unit of time.

Clause 4. The system of Clause 3, wherein the filling factor is determined according to the equation

$${K}_{f i l l} = \frac{{C}_{m e a s u r e d} - {C}_{min}}{{C}_{max} - {C}_{min}}$$

rein Cₘᵢₙ is the initial capacitance when the flexible infusion line is empty and Cₘₐₓ is the primed capacitance when the flexible infusion line is primed with the fluid, and wherein the volume is determined based on the equation V_{cycle}=Q_{const}×K_{fill}, wherein Q_{const} is a system-dependent constant conversion factor that accounts for properties of the flexible infusion line and a length of the flexible infusion line between the conductive plate and the conductor opposite the flexible infusion line.

Clause 5. The system of Clause 3, the one or more processors further configured to: determine the volume for a given cycle, the filling factor, and the flow rate when the hammer element is positioned at a low dead center during the delivery phase and the flexible infusion line compressed.

Clause 6. The system of any one of Clauses 1-5, wherein measuring the first capacitance comprises: sampling the capacitance over a revolution of the cam; and integrating the sampled capacitance over the revolution.

Clause 7. The system of any one of Clauses 1-6, the one or more processors further configured to: determine the volume for a plurality of cam cycles; and determine the flow rate based on the determined volume for each of the plurality of cam cycles over a given period.

Clause 8. The system of any one of Clauses 1-7, wherein the measured capacitance is based on a dielectric function comprising a dielectric constant associated with a gap between the conductive plate and the conductor opposite the flexible infusion line, a dielectric constant associated with the fluid that is filled within the flexible infusion line during the filling phase, and a dielectric constant associated with a material of the flexible infusion line.

Clause 9. The system of Clause 8, the one or more processors further configured to: receive an input of an infusion set identifier; and obtain the dielectric constant associated with the material based on the infusion set identifier; receive a fluid identifier; and obtain the dielectric constant associated with the fluid based on the fluid identifier.

Clause 10. The system of any one of Clauses 1-9, wherein the flexible infusion line being loaded in the pumping mechanism comprises the flexible infusion line being placed between the hammer element and a plate assembly in a door of the infusion pump, the plate assembly functioning as the conductor opposite the flexible infusion line, wherein the conductor opposite the flexible infusion line is grounded.

Clause 11. A method for measuring a flow rate of an infusion pump, comprising: causing at least one hammer element of a pumping mechanism of an infusion pump to move according to a cam motion to periodically compress an flexible infusion line loaded in a pumping mechanism of the infusion pump, according to a cycle speed, to fill the flexible infusion line with a fluid from a fluid reservoir during a filling phase of the cam motion and to deliver the fluid downstream of the hammer element during a delivery phase of the cam motion; measuring, during the cam motion, a first capacitance between a conductive plate coupled to the hammer element and a conductor opposite the flexible infusion line when the flexible infusion line is loaded in the pumping mechanism, between the conductive plate and the conductor; determining, based on the measured first capacitance, a volume of the fluid within the flexible infusion line during the filling phase; and determining a flow rate based on the determined volume and the cycle speed; and adjusting a speed of the infusion pump based on a difference between the determined flow rate and a programmed flow rate.

Clause 12. The method of Clause 11, further comprising: obtaining, from a lookup database, a fill factor based on the measured capacitance; and determining the volume based on a function of the fill factor and one or more predetermined variables.

Clause 13. The method of Clause 11, further comprising: measuring an initial capacitance when the flexible infusion line is empty; measuring a primed capacitance when the flexible infusion line is primed with the fluid; determining a filling factor based on a function of the measured first capacitance, the initial capacitance, and the primed capacitance; and determining the volume based on a function of the filling factor and one or more predetermined factors, wherein the cycle speed comprises a number of cam revolutions per unit of time.

Clause 14. The method of Clause 13, wherein the filling factor is determined according to the equation

$${K}_{f i l l} = \frac{{C}_{m e a s u r e d} - {C}_{min}}{{C}_{max} - {C}_{min}}$$

rein Cₘᵢₙ is the initial capacitance when the flexible infusion line is empty and Cₘₐₓ is the primed capacitance when the flexible infusion line is primed with the fluid, and wherein the volume is determined based on the equation V_{cycle}=Q_{const}×K_{fill}, wherein Q_{const} is a system-dependent constant conversion factor that accounts for properties of the flexible infusion line and a length of the flexible infusion line between the conductive plate and the conductor opposite the flexible infusion line.

Clause 15. The method of Clause 13, further comprising: determining the volume for a given cycle, the filling factor, and the flow rate when the hammer element is positioned at a low dead center during the delivery phase and the flexible infusion line compressed.

Clause 16. The method of any one of Clauses 11-15, wherein measuring the first capacitance comprises: sampling the capacitance over a revolution of a cam; and integrating the sampled capacitance over the revolution.

Clause 17. The method of any one of Clauses 11-16, further comprising: determining the volume for a plurality of cam cycles; and determining the flow rate based on the determined volume for each of the plurality of cam cycles over a given period.

Clause 18. The method of any one of Clauses 11-17, wherein the measured capacitance is based on a dielectric function comprising a dielectric constant associated with a gap between the conductive plate and the conductor opposite the flexible infusion line, a dielectric constant associated with the fluid that is filled within the flexible infusion line during the filling phase, and a dielectric constant associated with a material of the flexible infusion line.

Clause 19. The method of Clause 18, further comprising: receiving an input of an infusion set identifier; obtaining the dielectric constant associated with the material based on the infusion set identifier; receiving a fluid identifier; and obtaining the dielectric constant associated with the fluid based on the fluid identifier.

Clause 20. A non-transitory machine readable medium comprising instructions stored thereon that, when executed by a machine, causes the machine to perform a method according to any one of Clauses 11-20.

### Further Considerations

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms, web page and server. As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device, diagnostic device, monitoring device, or server in communication therewith.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A system for measuring a flow rate of an infusion pump, comprising:
at least one hammer element of a pumping mechanism of the infusion pump, the hammer element configured to move according to a cam motion of a cam associated with the pumping mechanism, to apply a periodic compression to a flexible infusion line when the flexible infusion line is loaded in the pumping mechanism;
a conductive plate coupled to the hammer element, between the hammer element and the flexible infusion line when the flexible infusion line is loaded in the pumping mechanism; and
one or more processors operatively coupled to the conductive plate and configured to:
cause the at least one hammer element to move according to the cam motion and a cycle speed to periodically compress the flexible infusion line to fill the flexible infusion line with a fluid from a fluid reservoir during a filling phase of the cam motion and to deliver the fluid downstream of the hammer element during a delivery phase of the cam motion;
measure, during the cam motion, a first capacitance between the conductive plate and a conductor opposite the flexible infusion line;
determine, based on the measured first capacitance, a volume of the fluid within the flexible infusion line during the filling phase;
determine a flow rate based on the determined volume and the cycle speed; and
adjust a speed of the infusion pump based on a difference between the determined flow rate and a programmed flow rate.

2. The system of claim 1, wherein the one or more processors are further configured to:
obtain, from a lookup database, a fill factor based on the measured capacitance; and
determine the volume based on a function of the fill factor and one or more predetermined variables.

3. The system of claim 1, the one or more processors further configured to:
measure an initial capacitance when the flexible infusion line is empty;
measure a primed capacitance when the flexible infusion line is primed with the fluid;
determine a filling factor based on a function of the measured first capacitance, the initial capacitance, and the primed capacitance; and
determine the volume based on a function of the filling factor and one or more predetermined factors,
wherein the cycle speed comprises a number of cam revolutions per unit of time.

4. The system of claim 3, wherein the filling factor is determined according to the equation
$${K}_{f i l l} = \frac{{C}_{m e a s u r e d} - {C}_{min}}{{C}_{max} - {C}_{min}}$$
rein Cₘᵢₙ is the initial capacitance when the flexible infusion line is empty and Cₘₐₓ is the primed capacitance when the flexible infusion line is primed with the fluid, and wherein the volume is determined based on the equation V_{cycle}=Q_{const}×K_{fill}, wherein Q_{const} is a system-dependent constant conversion factor that accounts for properties of the flexible infusion line and a length of the flexible infusion line between the conductive plate and the conductor opposite the flexible infusion line.

5. The system of claim 3, the one or more processors further configured to:
determine the volume for a given cycle, the filling factor, and the flow rate when the hammer element is positioned at a low dead center during the delivery phase and the flexible infusion line compressed.

6. The system of claim 1, wherein measuring the first capacitance comprises:
sampling the capacitance over a revolution of the cam; and
integrating the sampled capacitance over the revolution.

7. The system of claim 1, the one or more processors further configured to:
determine the volume for a plurality of cam cycles; and
determine the flow rate based on the determined volume for each of the plurality of cam cycles over a given period.

8. The system of claim 1, wherein the measured capacitance is based on a dielectric function comprising a dielectric constant associated with a gap between the conductive plate and the conductor opposite the flexible infusion line, a dielectric constant associated with the fluid that is filled within the flexible infusion line during the filling phase, and a dielectric constant associated with a material of the flexible infusion line.

9. The system of claim 8, the one or more processors further configured to:
receive an input of an infusion set identifier;
obtain the dielectric constant associated with the material based on the infusion set identifier;
receive a fluid identifier; and
obtain the dielectric constant associated with the fluid based on the fluid identifier.

10. The system of claim 1, wherein the flexible infusion line being loaded in the pumping mechanism comprises the flexible infusion line being placed between the hammer element and a plate assembly in a door of the infusion pump, the plate assembly functioning as the conductor opposite the flexible infusion line, wherein the conductor opposite the flexible infusion line is grounded.

11. A method for measuring a flow rate of an infusion pump, comprising:
causing at least one hammer element of a pumping mechanism of an infusion pump to move according to a cam motion to periodically compress an flexible infusion line loaded in a pumping mechanism of the infusion pump, according to a cycle speed, to fill the flexible infusion line with a fluid from a fluid reservoir during a filling phase of the cam motion and to deliver the fluid downstream of the hammer element during a delivery phase of the cam motion;
measuring, during the cam motion, a first capacitance between a conductive plate coupled to the hammer element and a conductor opposite the flexible infusion line when the flexible infusion line is loaded in the pumping mechanism, between the conductive plate and the conductor;
determining, based on the measured first capacitance, a volume of the fluid within the flexible infusion line during the filling phase;
determining a flow rate based on the determined volume and the cycle speed; and
adjusting a speed of the infusion pump based on a difference between the determined flow rate and a programmed flow rate.

12. The method of claim 11, further comprising:
obtaining, from a lookup database, a fill factor based on the measured capacitance; and
determining the volume based on a function of the fill factor and one or more predetermined variables.

13. The method of claim 11, further comprising:
measuring an initial capacitance when the flexible infusion line is empty;
measuring a primed capacitance when the flexible infusion line is primed with the fluid;
determining a filling factor based on a function of the measured first capacitance, the initial capacitance, and the primed capacitance; and
determining the volume based on a function of the filling factor and one or more predetermined factors,
wherein the cycle speed comprises a number of cam revolutions per unit of time.

14. The method of claim 13, wherein the filling factor is determined according to the equation
$${K}_{f i l l} = \frac{{C}_{m e a s u r e d} - {C}_{min}}{{C}_{max} - {C}_{min}}$$
rein Cₘᵢₙ is the initial capacitance when the flexible infusion line is empty and Cₘₐₓ is the primed capacitance when the flexible infusion line is primed with the fluid, and wherein the volume is determined based on the equation V_{cycle}=Q_{const}×K_{fill}, wherein Q_{const} is a system-dependent constant conversion factor that accounts for properties of the flexible infusion line and a length of the flexible infusion line between the conductive plate and the conductor opposite the flexible infusion line.

15. The method of claim 13, further comprising:
determining the volume for a given cycle, the filling factor, and the flow rate when the hammer element is positioned at a low dead center during the delivery phase and the flexible infusion line compressed.

16. The method of claim 1, wherein measuring the first capacitance comprises:
sampling the capacitance over a revolution of a cam; and
integrating the sampled capacitance over the revolution.

17. The method of claim 11, further comprising:
determining the volume for a plurality of cam cycles; and
determining the flow rate based on the determined volume for each of the plurality of cam cycles over a given period.

18. The method of claim 11, wherein the measured capacitance is based on a dielectric function comprising a dielectric constant associated with a gap between the conductive plate and the conductor opposite the flexible infusion line, a dielectric constant associated with the fluid that is filled within the flexible infusion line during the filling phase, and a dielectric constant associated with a material of the flexible infusion line.

19. The method of claim 18, further comprising:
receiving an input of an infusion set identifier;
obtaining the dielectric constant associated with the material based on the infusion set identifier;
receiving a fluid identifier; and
obtaining the dielectric constant associated with the fluid based on the fluid identifier.

20. A non-transitory machine readable medium comprising instructions stored thereon that, when executed by a machine, causes the machine to perform a method, comprising:
causing at least one hammer element of a pumping mechanism of an infusion pump to move according to a cam motion to periodically compress an flexible infusion line loaded in a pumping mechanism of the infusion pump, according to a cycle speed, to fill the flexible infusion line with a fluid from a fluid reservoir during a filling phase of the cam motion and to deliver the fluid downstream of the hammer element during a delivery phase of the cam motion;
measuring, during the cam motion, a first capacitance between a conductive plate coupled to the hammer element and a conductor opposite the flexible infusion line when the flexible infusion line is loaded in the pumping mechanism, between the conductive plate and the conductor;
determining, based on the measured first capacitance, a volume of the fluid within the flexible infusion line during the filling phase;
determining a flow rate based on the determined volume and the cycle speed; and
adjusting a speed of the infusion pump based on a difference between the determined flow rate and a programmed flow rate.
